(19) United States
(12) Patent Application Publication
(10) Pub. No.: US 2019/0029508 A1
(43) Pub. Date: Jan. 31, 2019

(54) ENDOSCOPE LIGHT SOURCE DEVICE, ENDOSCOPE, AND ENDOSCOPE SYSTEM

(71) Applicant: OLYMPUS CORPORATION, Tokyo (JP)

(72) Inventors: Motoki TABATA, Hino-shi (JP); Takeshi ITO, Hino-shi (JP)

(73) Assignee: OLYMPUS CORPORATION, Tokyo (JP)

(21) Appl. No.: 16/151,382

(22) Filed: Oct. 4, 2018

Related U.S. Application Data
(63) Continuation of application No. PCT/JP2016/061040, filed on Apr. 4, 2016.

(51) Int. Cl. A61B 1/06; A61B 1/00; G02B 23/24

(52) U.S. Cl. CPC A61B 1/0676; A61B 1/00165; A61B 1/04; A61B 1/00096; G02B 23/2461

(57) ABSTRACT

An endoscope light source device to which a connection portion of an endoscope is detachably attached includes a light source section that radiates primary light that enters an entrance end in the connection portion, a positioning member that positions the entrance end on a center axis of the primary light from the light source section when the connection portion is arranged in the light source device, and a pressing member that presses the connection portion toward the positioning member after the connection portion is arranged in the positioning member. The light source device further includes a heat transmission member that functions as at least one of the positioning member and the pressing member, and transmits heat generated from the connection portion.

### CROSS-REFERENCE TO RELATED APPLICATIONS

This application is a Continuation Application of PCT Application No. PCT/JP2016/061040, filed Apr. 4, 2016, the entire contents of which are incorporated herein by reference.

### BACKGROUND OF THE INVENTION

### 1. Field of the Invention

The present invention relates to an endoscope light source device, an endoscope, and an endoscope system.

### 2. Description of the Related Art

Recently, endoscope systems used in the medical field, for example, include the endoscope and the light source device. The endoscope includes an endoscope-side connector. The light source device includes a light source section that includes at least one of an LD, an LED, and a xenon lamp. When the endoscope-side connector is connected to the light source device, light radiated from the light source section enters an optical connection portion arranged in the endoscope-side connector. Then, the light is guided to a distal end of an endoscope insertion section from the optical connection portion, by a light guide provided in the endoscope from the optical connection portion to the distal end of the endoscope insertion section. The light is radiated toward an object from the distal end, and illuminates the object as illumination light.

Part of the light that enters the optical connection portion does not enter a core of the light guide, but is absorbed in a housing and the like of the optical connection portion and converted into heat. Thus, the optical connection portion is damaged by the heat. The heat is transmitted to the endoscope-side connector from the housing of the optical connection portion. Due to the heat, the temperature of the endoscope-side connector raises higher than an undesirable temperature to a user of the endoscope system or to the endoscope-side connector.

For example, Jpn. Pat. Appln. KOKAI Publication No. 2003-169776 discloses a configuration of preventing the optical connection portion from being damaged by the heat. The prevention configuration includes the protection tube covering the plastic fiber that is the light guide. The heat resistance of the protection tube is higher than that of the plastic fiber. The protection tube is covered with a metallic connection tube that is the housing of the optical connection portion. The protection tube prevents the plastic fiber from being connected to the connection tube, thereby preventing the heat of the connection tube from being transmitted to the plastic fiber and damaging the plastic fiber.

### BRIEF SUMMARY OF THE INVENTION

An aspect of the present invention is directed to an endoscope light source device to which an optical connection portion of an endoscope is detachably attached, the optical connection portion including an entrance end. The endoscope light source device comprises: a light source section that radiates primary light that enters the entrance end; a positioning member that is fixed to the endoscope light source device, and positions the entrance end on an optical axis that is a center axis of the primary light radiated from the light source section when the optical connection portion is arranged in the endoscope light source device; a pressing member that presses the optical connection portion toward the positioning member after the optical connection portion is arranged in the positioning member; and a first heat transmission member that is capable of functioning as at least one of the positioning member and the pressing member, and transmits heat generated from the optical connection portion when the optical connection portion is arranged in the endoscope light source device.

Another aspect of the present invention is directed to an endoscope that is detachably attached to the endoscope light source device described above.

Still another aspect of the present invention is directed to an endoscope system including the endoscope light source device described above and an endoscope detachably attached to the endoscope light source device.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing an endoscope system according to a first embodiment of the present invention;

FIG. 2 is a schematic view showing an endoscope including a single-line optical fiber, and a light source device;

FIG. 3A is a front view showing a state in which a first heat transmission member functioning as a pressing member is separated from a positioning member in a first orthogonal direction;

FIG. 3B is a front view showing a state in which the first heat transmission member functioning as the pressing member illustrated in FIG. 3A presses the optical connection portion on the positioning member illustrated in FIG. 3A;

FIG. 3C is a perspective view showing the first heat transmission member functioning as the pressing member illustrated in FIG. 3A, viewed from below;

FIG. 3D is a front view showing a state in which the first heat transmission member functioning as the pressing member illustrated in FIG. 3A presses the optical connection portion on a positioning member having a V-shaped cross section;

FIG. 3E is a front view showing a state in which the pressing member presses the optical connection portion on the first heat transmission member functioning as the positioning member;

FIG. 3F is a front view showing a state in which the first heat transmission member functioning as the pressing member presses the optical connection portion on the first heat transmission member functioning as the positioning member;

FIG. 4A is a side view showing a state in which the pressing member is switched to a released state by a switching mechanism;

FIG. 4B is a side view showing a state in which the pressing member is switched to a pressed state by the switching mechanism;

FIG. 5 is a schematic view showing an endoscope including a bundle fiber, and a light source device;

FIG. 6A is a schematic view showing a general endoscope-side connector, an optical connection portion, a light source device, a light source-side connection port, and a positioning member;

FIG. 6B is a front view showing the positioning member into which the optical connection portion is inserted;

FIG. 7 is a schematic view showing an endoscope and a light source device according to modification 1 of the first embodiment, and is a view showing an example of a heat transport mechanism;

FIG. 8 is a view showing another example of the heat transport mechanism according to modification 1 of the first embodiment;

FIG. 9A is a view showing another example of the heat transport mechanism according to modification 1 of the first embodiment, and is a schematic view showing a positioning member including a split sleeve and serving as a pressing member, and a fixing member;

FIG. 9 B is a cross-sectional view showing a periphery of the optical connection portion cut along a 9B-9B line illustrated in FIG. 9A;

FIG. 10 is a schematic view showing an endoscope and a light source device according to modification 2 of the first embodiment;

FIG. 11 is a schematic view showing an endoscope and a light source device according to a second embodiment;

FIG. 12A is a schematic view showing a first endoscope including a first bundle fiber and a light source device according to modification 1 of the second embodiment; and

FIG. 12B is a schematic view showing a second endoscope including a second bundle fiber and a light source device according to modification 1 of the second embodiment.

### DETAILED DESCRIPTION OF THE INVENTION

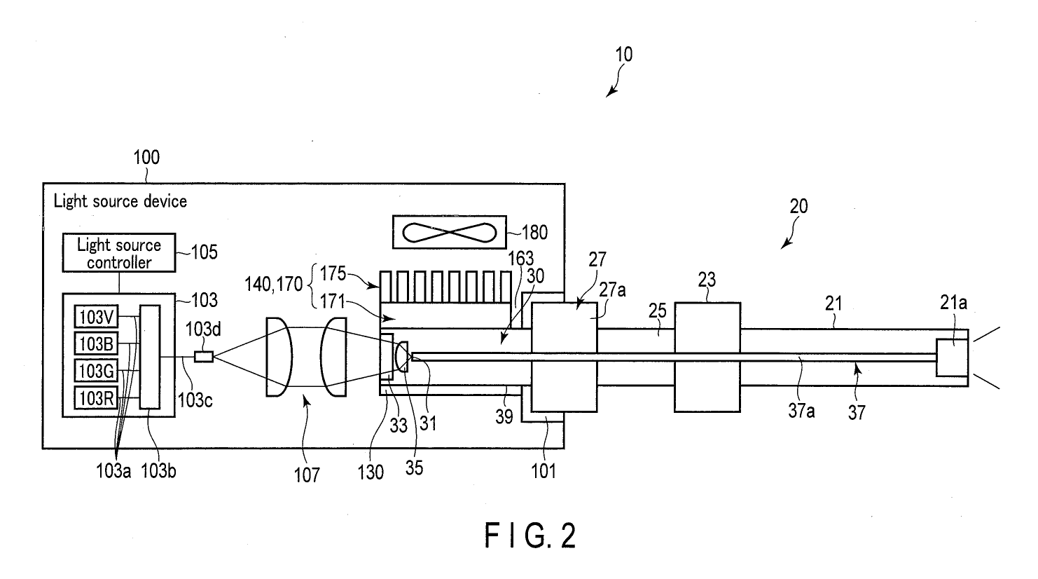

Hereinafter, embodiments of the present invention will be described in detail with reference to the drawings. In some drawings, the members are partly omitted for clarification of illustration, like omission of the switching mechanism 150 in FIG. 2, for example. A center axis of primary light radiated from an exit end 103d is referred to as an optical axis.

### First Embodiment

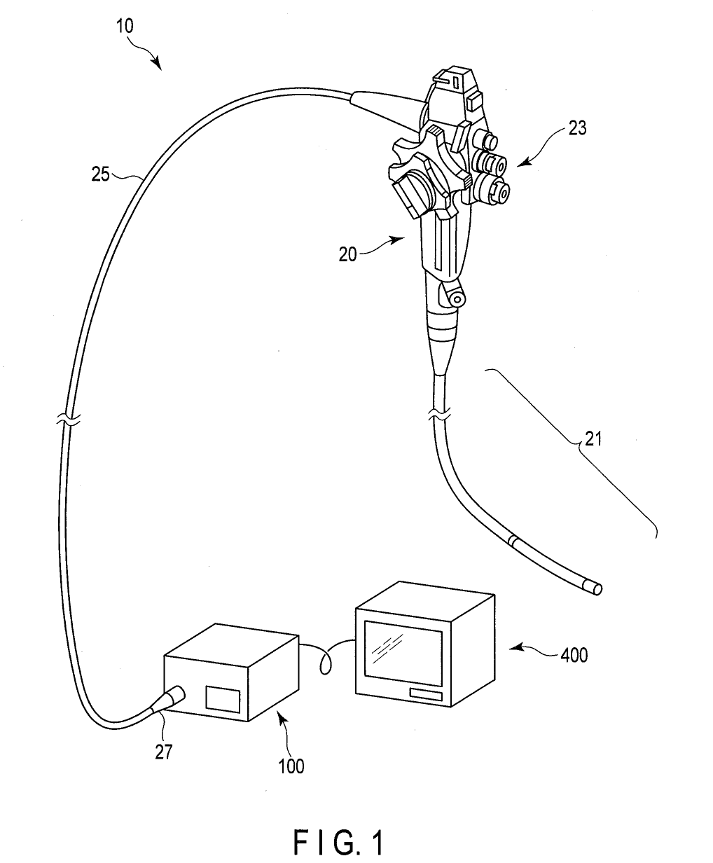

The first embodiment will be described with reference to FIG. 1 to FIG. 6B.

As illustrated in FIG. 1, an endoscope system 10 includes an endoscope 20 that images an object, an endoscope light source device (hereinafter, a light source device 100) to which the endoscope 20 is detachably attached, and a display 400 that is connected to the light source device 100 and displays an object image taken by the endoscope 20.

The display 400 displays an image taken by an imager (not shown) built in a distal end of an insertion section 21 arranged in the endoscope 20. The display 400 is a general display device, such as a liquid crystal display, a CRT display, or an organic EL display.

The endoscope 20 is an example of an insertion apparatus, for example, to be inserted into a conduit section and including an illuminating unit 21a (see FIG. 2). The endoscope 20 may be a direct-view-type endoscope or a side-view-type endoscope. The endoscope 20 of the present embodiment is described as an endoscope for medical use, for example, but is not limited to this. The endoscope 20 may be an endoscope for industrial use that is inserted into a conduit section of an industrial product such as a pipe, or may be an insertion apparatus such as a catheter including only the illuminator.

As illustrated in FIG. 1 and FIG. 2, the endoscope 20 includes a hollow and elongated insertion section 21 that is to be inserted into a conduit section, and a control section 23 that is connected to a proximal end of the insertion section 21 and controls the endoscope 20. The endoscope 20 includes a universal cord 25 extending from a side surface of the control section 23.

The insertion section 21 includes the illuminating unit 21a and an imager of an imaging unit (not shown). The illuminating unit 21a and the imaging unit are provided at the distal end of the insertion section 21.

The illuminating unit 21a desirably converts the optical characteristics of the primary light radiated from the light source device 100 to generate illumination light, radiating the illumination light outside. The illuminating unit 21a may convert light distribution characteristics of the primary light without converting the wavelength of the primary light. The illuminating unit 21a includes a scattering member including scattering particles. The illuminating unit 21a may include, for example, a wavelength conversion member (e.g., phosphor) that absorbs the primary light to emit fluorescent light (illumination light) having a wavelength different from the wavelength of the primary light. The illuminating unit 21a may include a diffusing member to diffuse the primary light without converting the wavelength of the primary light. The diffusing member may radiate diffusion light (illumination light) having a spread angle wider than that of the primary light and having low coherence.

The imager performs imaging by use of reflected light from the object illuminated with the illumination light. The imager includes, for example, a CCD imager or a CMOS imager. The imager outputs an electronic signal corresponding to the reflected light to an image processor (not shown) provided in the light source device 100 through a transmission path (not shown) that extends inside the insertion section 21, the control section 23, and the universal cord 25. The image processor processes the electronic signal, and causes the display 400 to display the image. The image processor is constituted by, for example, a hardware circuit including an ASIC or the like. The image processor may be constituted by a processor. If the image processor is constituted by a processor, a program code for causing the processor to function as an image processor by execution of the processor has been stored in an internal memory or an external memory (not shown) accessible by the processor. The image processor is incorporated in, for example, the light source device 100. The image processor may be configured separate from the light source device 100, and the display 400 may be connected to this.

As illustrated in FIG. 2, the universal cord 25 includes an endoscope-side connector 27 that is detachably attached to the light source-side connection port 101 functioning as a receptacle section of the light source device 100. The endoscope-side connector 27 is inserted into/removed from the light source-side connection port 101 for attachment and detachment.

The endoscope-side connector 27 is provided with the optical connection portion 30. The optical connection portion 30 is attached to/detached from the light source device 100 in accordance with insertion/removal of the endoscope-side connector 27 into/from the light source-side connection port 101. In the present embodiment, when the endoscope-side connector 27 is inserted into the light source-side connection port 101, the optical connection portion 30 is attached to the light source device 100. When the endoscope-side connector 27 is removed from the light source-side connection port 101, the optical connection portion 30 is detached from the light source device 100. When the endoscope-side connector 27 is connected to the light source-side connection port 101, the optical connection portion 30 is optically connected to the light source section 103 of the light source device 100. The optical connection portion 30 includes an entrance end 31 that the primary light enters, and the optical connection portion 30 is arranged in the endoscope 20, and detachably attached to the light source device 100.

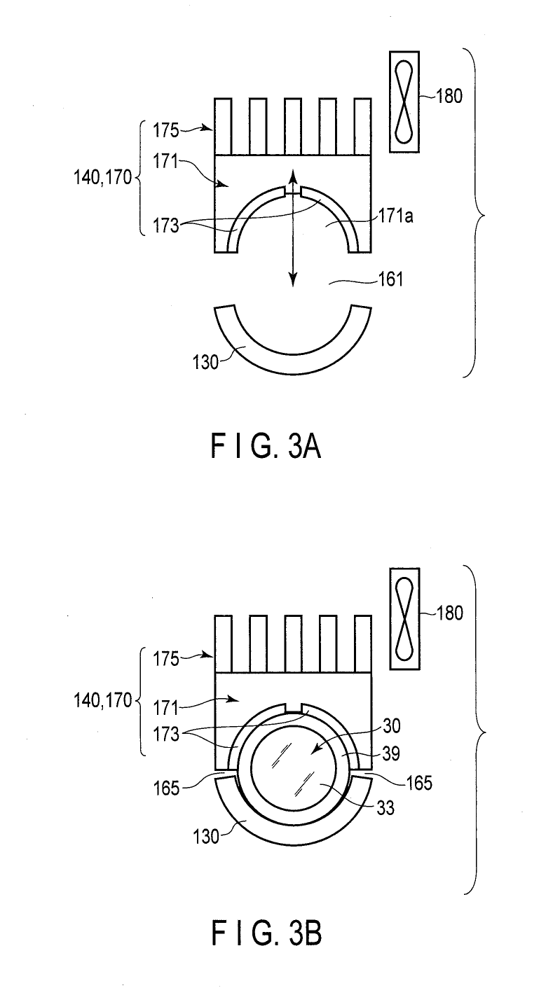

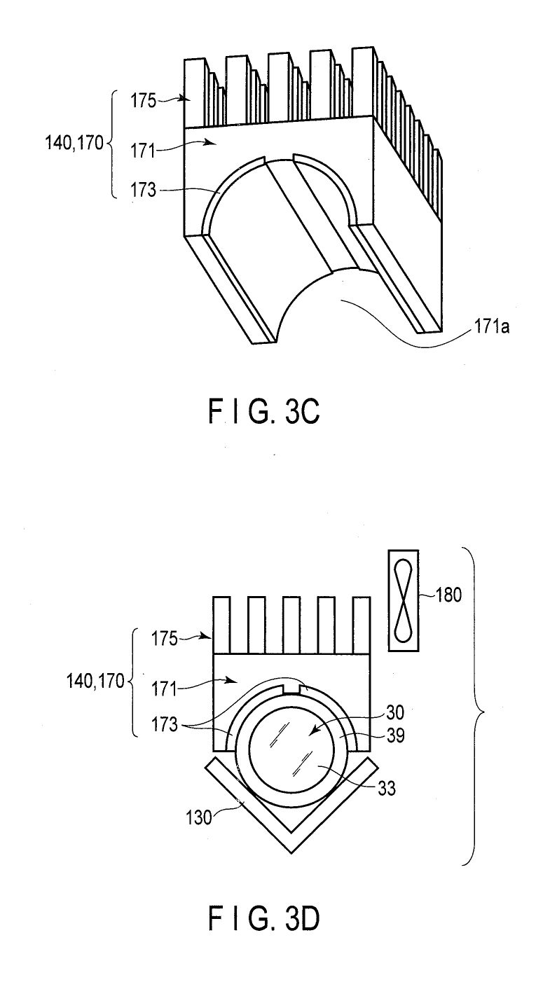

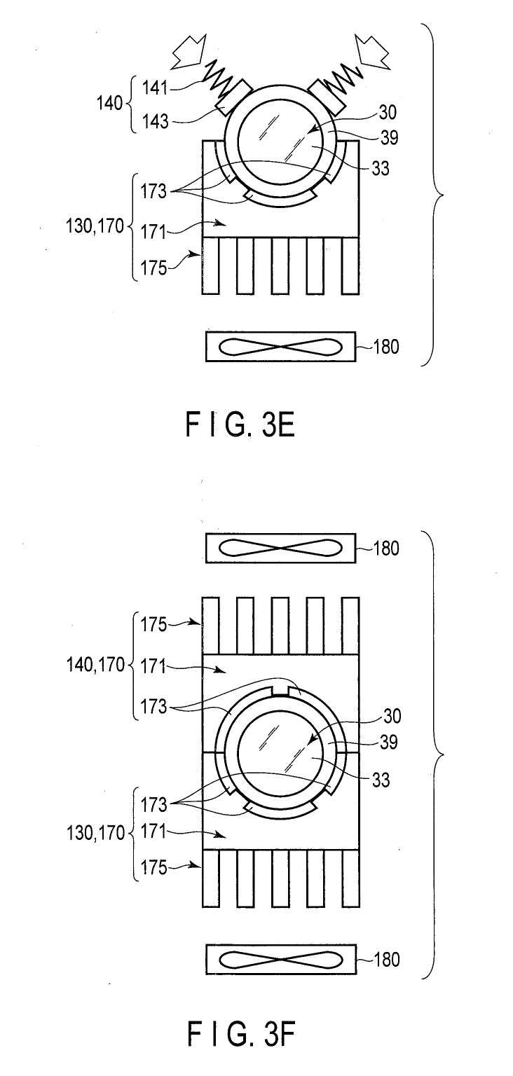

The optical connection portion 30 includes a cover glass 33, a lens 35, an end portion of a light guide 37 including the entrance end 31, and a housing 39 accommodating the cover glass 33, the lens 35, and the end portion of the light guide 37. The cover glass 33 is arranged on the end surface of the housing 39, and protects the entrance end 31 and the lens 35. The cover glass 33 is a member through which the primary light can be transmitted. For example, the cover glass 33 may be transparent. The lens 35 collects, at the entrance end 31, the primary light that has passed through the cover glass 33. The light guide 37 is arranged inside the endoscope-side connector 27, the universal cord 25, the control section 23, and the insertion section 21, from the housing 39. The exit end of the light guide 37 is optically connected to the illuminating unit 21a. The light guide 37 guides the primary light from the entrance end 31 to the illuminating unit 21a. The light guide 37 includes, for example, a single-line optical fiber 37a. The housing 39 is attached to an endoscope-side housing 27a of the endoscope-side connector 27. As illustrated in FIG. 3B and FIG. 3D, the housing 39 has a tubular shape, for example, a cylindrical shape. The housing 39 includes, for example, a member having high heat conductivity. The member is, for example, a metallic member, such as copper, aluminum, SUS, stainless steel, and aluminum nitride.

As illustrated in FIG. 2, the light source device 100 includes the light source-side connection port 101 to which the endoscope-side connector 27 is detachably connected, the light source section 103 that radiates the primary light, the light source controller 105 that controls the light source section 103, and a light-collecting optical system 107 that collects the primary light in the lens 35. When the endoscope-side connector 27 is connected to the light source-side connection port 101, the light source device 100 can bring the primary light into the endoscope 20.

The light source section 103 include light sources 103V, 103B, 103G, and 103R, and light guides 103a, the number of which is equal to the light sources 103V, 103B, 103G, and 103R and optically connected to the respective light sources. The light source section 103 further includes a combiner 103b that combines the light guided by the respective light guides 103a as the primary light, and a light guide 103c that guides the primary light combined by the combiner 103b.

The light sources 103V, 103B, 103G, and 103R radiate light having high light intensity at a collected point, for example.

The light source 103V includes, for example, a laser diode that emits purple laser light. The center wavelength of the laser light is, for example, 405 nm.

The light source 103B includes, for example, a laser diode that emits blue laser light. The center wavelength of the laser light is, for example, 445 nm.

The light source 103G includes, for example, a laser diode that emits green laser light. The center wavelength of the laser light is, for example, 532 nm.

The light source 103R includes, for example, a laser diode that emits red laser light. The center wavelength of the laser light is, for example, 635 nm.

The light sources 103V, 103B, 103G, and 103R may each include a xenon lamp or an LED, for example. The number of the light sources, the colors of the light radiated from the light sources, and the center wavelengths of the light are not particularly limited.

The light guides 103a and 103c each include, for example, a single-line optical fiber.

The light source controller 105 controls quantities of emitted light of the light sources 103V, 103B, 103G, and 103R, based on input information that is input into an input device (not shown) by the user of the endoscope system 10. By controlling the quantities of emitted light of the light sources 103V, 103B, 103G, and 103R, the color of the primary light is desirably adjusted. For instance, if the respective the quantities of light of the light sources 103V, 103B, 103G, and 103R are controlled at desirable ratios, the primary light becomes white light. The primary light is radiated from the exit end 103d of the light guide 103c toward the light-collecting optical system 107.

The input device is a general input device, for example, a keyboard, a pointing device such as a mouse, a tag reader, a button switch, a slider, and a dial. The input device may be used, for example, for inputting instructions on observation modes, such as a white light observation using the light sources 103B, 103G, and 103R, and a specific light observation using the light sources 103V and 103G. The input device is used for inputting various instructions for operating the endoscope system 10 by the user. The combiner 103b includes, for example, an optical fiber combiner. The light source controller 105 is constituted by, for example, a hardware circuit including an ASIC or the like. The light source controller 105 may be constituted by a processor. If the light source controller 105 is constituted by a processor, a program code for causing the processor to function as the light source controller 105 by execution of the processor has been stored in an internal memory or an external memory (not shown) accessible by the processor.

The light-collecting optical system 107 includes lenses. The light-collecting optical system 107 collects light on the lens 35 so as to cause the primary light radiated from the exit end 103d to enter the entrance end 31.

As illustrated in FIG. 2, the light source device 100 includes a positioning member 130 that positions the entrance end 31 on the optical axis, which is the center axis of the primary light radiated from the exit end 103d of the light source section 103, and a pressing member 140 that presses the optical connection portion 30 on at least part of the positioning member 130 when the optical connection portion 30 is arranged in the light source device 100.

The positioning member 130 is fixed inside the light source device 100 in a state in which the positioning member 130 is accurately positioned with respect to the exit end 103d and the light-collecting optical system 107. The positioning member 130 is arranged further backward than the light source-side connection port 101 in the insertion direction of the endoscope-side connector 27. The positioning member 130 is continuous with the light source-side connection port 101. The positioning member 130 positions the entrance end 31 so that the entrance end 31 is positioned (optically connected) with respect to the exit end 103d. For example, the positioning member 130 positions the entrance end 31 so that the entrance end 31 is arranged on the same straight line as the exit end 103d.

As illustrated in FIG. 2 and FIG. 3B, the positioning member 130 is brought into surface contact with the optical connection portion 30. The surface contact indicates that the entire peripheral surface of the positioning member 130 facing the outer peripheral surface of the housing 39 of the optical connection portion 30 comes into contact with the entire outer peripheral surface of the housing 39, for example. Therefore, in the present embodiment, the positioning member 130 has, for example, a substantially semi-cylindrical shape. The inner shape and the inner diameter of the positioning member 130 are substantially equal to the outer shape and the outer diameter of the housing 39. The length of the positioning member 130 is preferably substantially equal to the length of the optical connection portion 30.

The positioning member 130 may be, for example, a support member that receives and supports the optical connection portion 30. Therefore, the shape of the positioning member 130 may correspond to the shape of the housing 39. Alternatively, as illustrated in FIG. 3D, the positioning member 130 may have a V-shaped cross section, for example. In this case, in the cross section, the positioning member 130 can always come into point contact with the cylindrical housing 39 at two portions. The point contact indicates, for example, that the contact is made in a narrower range than the surface contact, and that part of the peripheral surface of the positioning member 130 comes into contact with part of the outer peripheral surface of the housing 39. Thus, the positioning member 130 having the V-shaped cross section can stably position as compared to when the positioning section has a semi-cylindrical shape.

In this way, the positioning member 130 is brought into surface contact or point contact with the optical connection portion 30.

When the endoscope-side connector 27 is inserted into/removed from the light source-side connection port 101, the housing 39 can slide on the positioning member 130 in the longitudinal axis direction of the housing 39. It is preferable that the outer peripheral surface of the housing 39 and the inner peripheral surface of the positioning member 130 are smooth.

It is preferable that the positioning member 130 is, for example, a member having high rigidity such as metal. The positioning member 130 is, for example, stainless steel. The positioning member 130 may include, for example, a member having high heat conductivity. The member is, for example, a metallic member, such as copper, aluminum, SUS, stainless steel, and aluminum nitride.

The positioning member 130 positions the optical connection portion 30 through the housing 39. For example, when the optical connection portion 30 is arranged on the positioning member 130, the positioning member 130 positions the optical connection portion 30 at a lower side in the radial direction of the optical connection portion 30. The lower side is a lower side in the vertical direction in FIG. 2. Positioning at an upper side in the radial direction of the optical connection portion 30 is carried out by pinching described later. The upper side is an upper side in the vertical direction in FIG. 2. Positioning in the longitudinal axis direction of the optical connection portion 30 is carried out, for example, by the endoscope-side connector 27 being caught by the light source-side connection port 101. Positioning in the longitudinal axis direction of the optical connection portion 30 may be carried out by a stopper portion (not shown) arranged in the positioning member 130. The stopper portion is brought into contact with one end surface of the housing 39 to carry out the positioning.

When the endoscope-side connector 27 is removed from the light source-side connection port 101, as illustrated in FIG. 3A, the pressing member 140 faces the positioning member 130 in the first orthogonal direction orthogonal to the optical axis direction. The optical axis direction indicates a direction in which the optical connection portion 30 is inserted into/removed from the light source device 100. A first space 161 is arranged between the pressing member 140 and the positioning member 130. The first space 161 is larger than the optical connection portion 30. The pressing member 140 is separated from the positioning member 130.

When the endoscope-side connector 27 is inserted into the light source-side connection port 101, as illustrated in FIG. 3B, the pressing member 140 comes into contact with the outer peripheral surface of the housing 39 in the first orthogonal direction, and presses the optical connection portion 30 toward the positioning member 130. As illustrated in FIG. 2, the pressing member 140 presses the optical connection portion 30 toward the positioning member 130 over the entire length of the optical connection portion 30. In this state, the optical connection portion 30 is pinched between the pressing member 140 and the positioning member 130 in the first orthogonal direction. The entire inner peripheral surface of the positioning member 130 is brought into close contact with the outer peripheral surface of the optical connection portion 30. An inner peripheral surface of a recessed portion 171a described later of the pressing member 140 is brought into close contact with the outer circumferential surface of the optical connection portion 30. Thus, the optical connection portion 30 is positioned at an upper side in the radial direction of the optical connection portion 30. The optical connection portion 30 is pinched between the positioning member 130 and the pressing member 140, and is thus positioned on the optical axis.

In the present embodiment, the pressing member 140 is movable in the first orthogonal direction so that when the endoscope-side connector 27 is inserted into the light source-side connection port 101, the pressing member 140 comes closer to the positioning member 130, and when the endoscope-side connector 27 is removed from the light source-side connection port 101, the pressing member 140 is separated toward the positioning member 130. That is, the pressing member 140 moves along the first orthogonal direction in conjunction with insertion/removal of the endoscope-side connector 27 into/from the light source-side connection port 101. The pressing member 140 is switched between a pressed state and a non-pressed state in conjunction with insertion/removal of the endoscope-side connector 27 into/from the light source-side connection port 101.

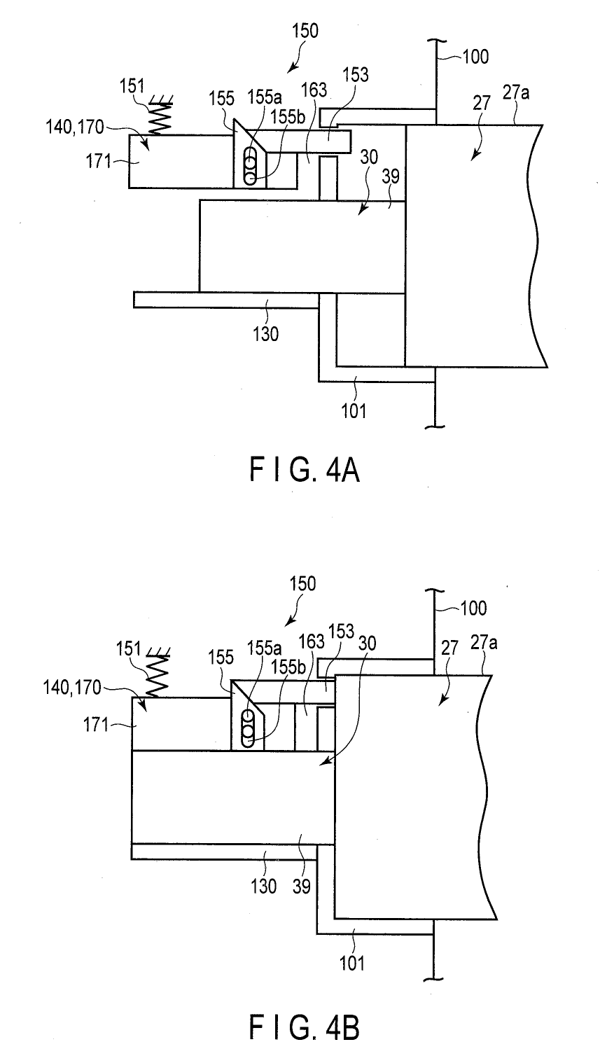

The light source device 100 includes a switching mechanism 150 that switches between a pressed state in which the pressing member 140 presses the optical connection portion 30 toward the positioning member 130 when the optical connection portion 30 is attached to the light source device 100 as illustrated in FIG. 4B, and a released state in which the pressing member 140 releases the pressing against the optical connection portion 30 when the optical connection portion 30 is removed from the light source device 100 as illustrated in FIG. 4A. For clarification of illustration, illustration of the switching mechanism 150 is omitted in the drawings other than FIG. 4A and FIG. 4B. The switching mechanism 150 moves the pressing member 140 in the first orthogonal direction in conjunction with insertion/removal of the endoscope-side connector 27 into/from the light source-side connection port 101. The switching mechanism 150 is a link mechanism that converts the insertion force of the endoscope-side connector 27 to the light source-side connection port 101 into the pressing force in the first orthogonal direction. The insertion force acts in the longitudinal axis direction of the optical connection portion 30. In the following, an example of the switching mechanism 150 will be briefly described. The switching mechanism 150 is not limited to the configuration described below, and may be moved by the pressing of a spring (not shown) or the like.

The switching mechanism 150 includes a pulling member 151, a pressing slider 153, and a driven slider 155.

The pulling member 151 has an end fixed to a later-described heat transmission main body 171 in the pressing member 140, and the other end fixed to the housing of the light source device 100. The pulling member 151 has a spring that extends and contracts in the first orthogonal direction in conjunction with insertion/removal of the endoscope-side connector 27 into/from the light source-side connection port 101. When the endoscope-side connector 27 is removed from the light source-side connection port 101, the pulling member 151 is shortened so that the pressing member 140 is away from the positioning member 130 in the first orthogonal direction, thereby pulling the pressing member 140 upward in the first orthogonal direction. When the endoscope-side connector 27 is inserted into the light source-side connection port 101, the pulling member 151 can extend.

The pressing slider 153 and the driven slider 155 are, for example, plate materials. The pressing slider 153 moves along the longitudinal axis direction of the optical connection portion 30 in conjunction with insertion/removal of the endoscope-side connector 27 into/from the light source-side connection port 101. The pressing slider 153 is arranged on the side of the heat transmission main body 171. The driven slider 155 moves along the first orthogonal direction in conjunction with insertion/removal of the endoscope-side connector 27 into/from the light source-side connection port 101. The driven slider 155 is arranged on the side of the heat transmission main body 171. The driven slider 155 has a through hole 155b through which a plurality of fixed pins 155a pass. The fixed pins 155a are attached to the side surface of the heat transmission main body 171. The fixed pins 155a and the through hole 155b constitute a guide that guide the movement of the driven slider 155 in the first orthogonal direction.

When the endoscope-side connector 27 is inserted into the light source-side connection port 101, the pressing slider 153 is pressed in the insertion direction of the endoscope-side connector 27 by the endoscope-side connector 27, thereby moving toward the driven slider 155 along the longitudinal axis direction of the optical connection portion 30. The pressing slider 153 presses the driven slider 155 toward the lower side (positioning member 130) in the first orthogonal direction. This pressing generates a first pressing force directed downward in the first orthogonal direction. When the first pressing force equal to or higher than the pulling force of the pulling member 151 is applied to the driven slider 155, the driven slider 155 moves downward, and the edge of the through hole 155b presses the fixed pins 155a downward. Then, the pulling member 151 extends, and the pressing member 140 to which the fixed pins 155a are fixed moves toward the optical connection portion 30 by the first pressing force. The pressing member 140 presses the optical connection portion 30 toward the positioning member 130. When the endoscope-side connector 27 is engaged with the light source-side connection port 101, the first pressing force equal to or higher than the pulling force is maintained. Thus, the pressing member 140 continues to press the optical connection portion 30 toward the positioning member 130.

When the endoscope-side connector 27 is removed from the light source-side connection port 101, the first pressing force is eliminated, and the pulling force acts on the pressing member 140. Accordingly, the pulling member 151 contracts and pulls the pressing member 140. As a result, the pressing member 140 moves upward in the first orthogonal direction, and moves to be away from the positioning member 130. The fixed pins 155a of the heat transmission main body 171 move the driven slider 155 upward in the first orthogonal direction through the through hole 155b. The driven slider 155 presses the pressing member 140 in the removal direction of the endoscope-side connector 27. This pressing generates a second pressing force toward the removal direction of the endoscope-side connector 27. The pressing member 140 returns to an initial position as illustrated in FIG. 4A by the second pressing force. The initial position is a position at which the endoscope-side connector 27 can press the pressing member 140 when the endoscope-side connector 27 is inserted into the light source-side connection port 101. At the initial position, the end portion of the pressing member 140 is positioned inside the light source-side connection port 101 so that the endoscope-side connector 27 can press the end portion.

The pressing member 140 is arranged away from the light source-side connection port 101 in the longitudinal axis direction of the optical connection portion 30, in order to prevent the pressing member 140 and the light source-side connection port 101 from wearing against each other by the movement of the pressing member 140. Therefore, as illustrated in FIG. 2, FIG. 4A, and FIG. 4B, a second space 163 is formed between the pressing member 140 and the light source-side connection port 101 in the longitudinal axis direction of the optical connection portion 30. The length of the pressing member 140 is, for example, substantially equal to the length of the positioning member 130, and is shorter than the length of the optical connection portion 30.

The light source device 100 includes a first heat transmission member 170 that is capable of functioning as at least one of the positioning member 130 and the pressing member 140 and transmits heat generated from the optical connection portion 30 when the optical connection portion 30 is arranged in the light source device 100. The first heat transmission member 170 releases the heat to a peripheral space of the first heat transmission member 170 from the optical connection portion 30. The peripheral space of the first heat transmission member 170 is included in the peripheral space of the optical connection portion 30.

As the first example of the first heat transmission member 170, an example in which the first heat transmission member 170 functions as the pressing member 140 will be described below, with reference to FIG. 2, FIG. 3A, FIG. 3B, FIG. 3C, and FIG. 3D.

The first heat transmission member 170 includes the heat transmission main body 171, a contact portion 173, and a first heat release portion 175. The contact portion 173 transmits the heat generated from the optical connection portion 30 to the heat transmission main body 171. The heat transmission main body 171 transmits the heat transmitted from the contact portion 173 to the first heat release portion 175. The first heat release portion 175 is thermally and indirectly connected to the contact portion 173 through the heat transmission main body 171. The first heat release portion 175 releases the heat transmitted from the contact portion 173 through the heat transmission main body 171 to the peripheral space of the first heat release portion 175. The peripheral space of the first heat release portion 175 is included in the peripheral space of the first heat transmission member 170.

The heat transmission main body 171 is interposed between the contact portion 173 and the first heat release portion 175 in the heat movement path, and supports the contact portion 173 and the first heat release portion 175.

As illustrated in FIG. 3B, the cross section of the heat transmission main body 171 is, for example, a semi-cylindrical recess. Third spaces 165 are provided in the cross section between the heat transmission main body 171 and the positioning member 130 at the time of pressing. The third spaces 165 prevent the heat transmission main body 171 and the positioning member 130 from wearing against each other due to the movement of the heat transmission main body 171. Thus, at the time of pressing, the heat transmission main body 171 is not continuous with the positioning member 130, and is arranged away from the positioning member 130. The third spaces 165 are arranged between the edges of the heat transmission main body 171 and both ends of the positioning member 130, in the cross section between heat transmission main body 171 and the positioning member 130 and the first orthogonal direction. For the third spaces 165, the length of the recessed portion 171a in the circumferential direction of the recessed portion 171a (see FIG. 3A) of the heat transmission main body 171 and the length of the positioning member 130 in the circumferential direction of the positioning member 130 are set in an appropriate and desirable manner.

The inner shape and the inner diameter of the heat transmission main body 171 representing the inner shape and the inner diameter of the recessed portion 171a are substantially equal to the outer shape and the outer diameter of the housing 39. The shape of the recessed portion 171a may correspond to the shape of the housing 39. The heat transmission main body 171 includes, for example, a member having high heat conductivity. The member is, for example, a metallic member, such as copper, aluminum, SUS, stainless steel, and aluminum nitride.

The contact portion 173 is, for example, a sheet-like member. The contact portion 173 is arranged on the inner peripheral surface of the recessed portion 171a that functions as a contact part with respect to the optical connection portion 30 in the first heat transmission member 170. The inner peripheral surface is a surface facing the optical connection portion 30. The contact portion 173 is divided into two so that part of the inner peripheral surface of the recessed portion 171a is exposed. Each of the contact portions 173 is preferably arranged over the entire length of the inner peripheral surface in the longitudinal axis direction of the inner peripheral surface. The exposed portion is arranged linearly in the longitudinal axis direction of the heat transmission main body 171. The contact portion 173 has a desired thickness. In the cross section of the heat transmission main body 171, the end of the contact portion 173 is arranged on the same plane as the edge of the recessed portion 171a.

The contact portion 173 includes, for example, a member having high heat conductivity. The member has, for example, a resin material, and a filler mixed with the resin material. The resin material is, for example, silicone, acrylic, polyphylene, or the like. The filler is, for example, a metal filler, a ceramic filler, or the like.

As illustrated in FIG. 3B, when the optical connection portion 30 is arranged in the light source device 100 and the pressing member 140 functioning as the first heat transmission member 170 presses the optical connection portion 30 toward the positioning member 130, the contact portion 173 comes directly into contact with the optical connection portion 30. Thus, the contact portion 173 is thermally and directly connected to the optical connection portion 30. At this time, the contact portion 173 can be deformed with respect to the optical connection portion 30, specifically in conformity with the shape of the outer peripheral surface of the housing 39. Therefore, the entire surface of the contact portion 173 can be directly brought into close contact with the optical connection portion 30, specifically the outer peripheral surface of the housing 39. The contact portion 173 is brought into surface contact with the optical connection portion 30, and the heat is transmitted from the optical connection portion 30. The contact portion 173 functions as a pad.

For example, the first heat release portion 175 is arranged on the side opposite to the contact portion 173 with the heat transmission main body 171 therebetween, in the first orthogonal direction. The first heat release portion 175 may be arranged on the outer peripheral surface of the heat transmission main body 171. The first heat release portion 175 may function as a heat sink, for example. The first heat release portion 175 includes, for example, rod-shaped metal members. An example of the metal members includes aluminum. The metal members are, for example, provided vertically along the first orthogonal direction. The metal members are, for example, arranged at equal intervals with respect to each other in the insertion/removal direction, and a second orthogonal direction orthogonal to the insertion/removal direction and the first orthogonal direction. The metal members increase the surface area of the first heat release portion 175. Therefore, the heat release ability of the first heat release portion 175 improves, and the heat transmission efficiency from the first heat release portion 175 to the peripheral space of the first heat release portion 175 improves. The first heat release portion 175 may have a plate shape.

As illustrated in FIG. 2, the light source device 100 includes a first cooler 180. In the present embodiment, the first cooler 180 includes, for example, an air cooler such as a fan that provides wind toward the first heat release portion 175. The first cooler 180 further improves the heat transmission efficiency of the heat from the first heat release portion 175 to the peripheral space of the first heat release portion 175. In FIG. 2, the first cooler 180 is arranged above the first heat release portion 175 in the first orthogonal direction. However, the arrangement position of the first cooler 180 can be appropriately changed as desired, for example, in accordance with the heat release ability of the first heat release portion 175, the arrangement state of the components inside the light source device 100, and the like. As illustrated in FIG. 3A, FIG. 3B, and FIG. 3D, the first cooler 180 may be arranged on the side of the first heat transmission member 170, for example. The first cooler 180 may be arranged so that the wind flows through gaps (not shown) among the metal members of the first heat release portion 175. The first cooler 180 may blow air toward the heat transmission main body 171 or the optical connection portion 30 as well as toward not only the first heat release portion 175.

For example, the first cooler 180 is driven when the endoscope-side connector 27 is inserted into the light source-side connection port 101, and is stopped when the endoscope-side connector 27 is removed from the light source-side connection port 101. The first cooler 180 may be controlled by the light source controller 105 so as to be driven when the light source section 103 is driven, in other words, when the light source section 103 radiates the primary light, and stopped when the light source section 103 stops.

Natural cooling may be carried out with omission of the first cooler 180 as long as the heat release ability of the first heat release portion 175 is secured.

As the second example of the first heat transmission member 170, an example, in which the first heat transmission member 170 functions as the positioning member 130, will be described below, with reference to FIG. 3E.

In this case, the pressing member 140 includes a spring 141 that extends and contracts, and a pad 143 that is arranged at an end of the spring 141 and is in close contact with the outer peripheral surface of the housing 39. The other end of the spring 141 is connected to the housing of the light source device 100. When the pressing member 140 presses the optical connection portion 30 toward the positioning member 130, the pad 143 can be deformed so that the pad 143 comes into close contact with the outer peripheral surface of the housing 39. The pad 143 can be deformed in conformity with the shape of the outer peripheral surface. The pad 143 may function as a contact portion. The arrangement position and the number of the pressing member 140 are not particularly limited.

The configuration of the first heat transmission member 170 functioning as the positioning member 130 is substantially the same as that in the first example. It is preferable that the contact portion 173 is arranged coaxially with the pad 143. As a result, the pressing force of the pressing member 140 acts on the contact portion 173 without a waste through the optical connection portion 30. Then, the optical connection portion 30 is reliably pressed against the contact portion 173 to be brought into contact with the contact portion 173, and thermally and directly brought into contact with the contact portion 173. For this reason, the heat generated from the optical connection portion 30 is transmitted to the first heat transmission member 170.

The first cooler 180 is, for example, arranged below the first heat release portion 175 in the orthogonal direction. The first cooler 180 may be arranged on the side of the first heat transmission member 170 in substantially the same manner as in the first example. Furthermore, the first cooler 180 may be arranged above or on the side of the optical connection portion 30 in the orthogonal direction.

As the third example of the first heat transmission member 170, an example, in which the first heat transmission member 170 functions as the positioning member 130 and the pressing member 140, will be described below, with reference to FIG. 3F.

The configuration of the first heat transmission member 170 functioning as the pressing member 140 is substantially the same as that of the first example; the configuration of the first heat transmission member 170 functioning as the positioning member 130 is substantially the same as that in the second example. The arrangement position of the first cooler 180 is also substantially the same as that in the first and second examples.

In this manner, the first heat transmission member 170 only has to function as at least one of the positioning member 130 and the pressing member 140.

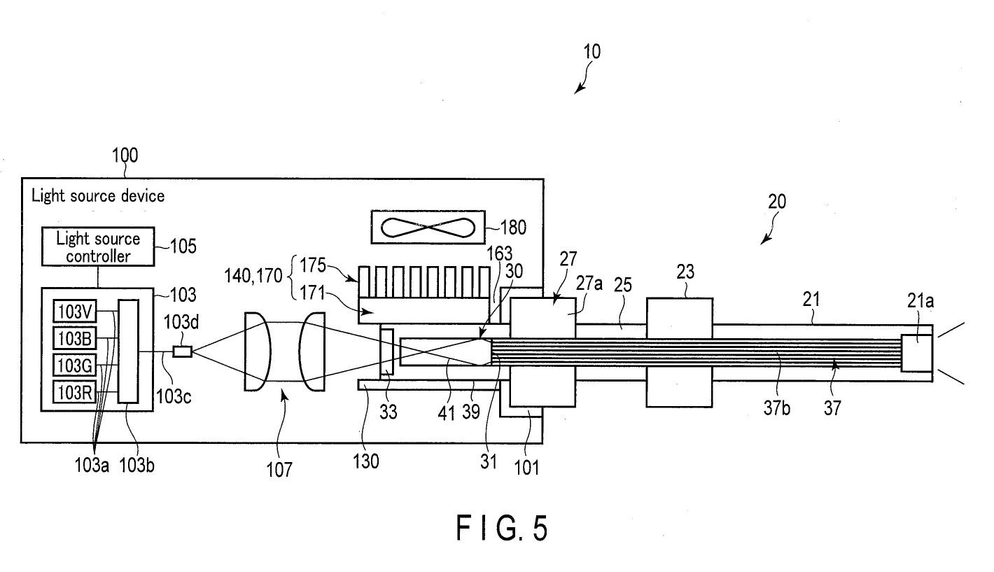

The light source device 100 needs to be shared and standardized for various types of endoscopes 20 having different optical functions, for example, and it is necessary to be a general device. Therefore, the first heat transmission member 170 needs to correspond to each optical function. The optical function refers to, for example, characteristics of the light guide 37. Here, as an example of the optical function, an explanation will be given using the bundle fiber 37b that is an example of the light guide 37 shown in FIG. 5.

In this case, the optical connection portion 30 includes an entrance end 31, a cover glass 33, a rod lens 41, an end portion of the light guide 37 including the entrance end 31, and a housing 39 accommodating the cover glass 33, the rod lens 41, and the end portion of the light guide 37. The rod lens 41 allows the light intensity to be uniform at the entrance end 31. In general, the light intensity of the laser light, which is the primary light, is strong at the central portion of a beam of laser light, and is weak as it goes away from the center portion. Accordingly, the light intensity of the beam of laser light is non-uniform. If the beam of laser light directly enters the bundle fiber 37b from the entrance end 31 in this state, the quantity of light that enters each optical fiber of the bundle fiber 37b varies. The tendency of variations propagates to the end portion (illuminating unit 21a) of the bundle fiber 37b. As a result, the light intensity of the beam of laser light radiated from the bundle fiber 37b is biased, and luminance unevenness or light distribution unevenness occurs as illumination light. However, since the rod lens 41 repeatedly reflects the laser light as the primary light within the rod lens 41, the laser light enters the entire entrance end 31 substantially uniformly. For this reason, the deviation of the light intensity is eliminated, so that the light intensity of the laser light becomes uniform. Therefore, luminance unevenness or light distribution unevenness is prevented.

If the bundle fiber 37b is provided, the position of the entrance end 31 of the bundle fiber 37b with respect to the exit end 103d (see FIG. 5) is different from the position of the entrance end 31 of the single-line optical fiber with respect to the exit end 103d (see FIG. 2). Therefore, the optical connection portion 30 having the bundle fiber 37b shown in FIG. 5 is shorter than the optical connection portion 30 having the single-line optical fiber 37a shown in FIG. 2. According to the length of the optical connection portion 30, the first heat transmission member 170 shown in FIG. 5 is shorter than the first heat transmission member 170 shown in FIG. 2.

Regardless of the single-line optical fiber 37a or the bundle fiber 37b, since the primary light is collected at the entrance end 31, the entrance end 31 is the portion where the heat is mostly generated in the optical connection portion 30. Therefore, considering the heat transmission from the entrance end 31 to the first heat transmission member 170, the first heat transmission member 170 only has to have a length of the optical connection portion 30, in other words, only has to be arranged at least at the periphery of the entrance end 31 including the entrance end 31 regardless of where the entrance end 31 is arranged with respect to the exit end 103d. Specifically, the contact portion 173 of the first heat transmission member 170 only has to be thermally connected to the entrance end 31. Therefore, the periphery indicates an area in which the contact portion 173 of the first heat transmission member 170 can be thermally connected to the entrance end 31. In this manner, the first heat transmission member 170 does not need to be arranged directly at the entrance end 31, but is arranged, for example, in the housing 39 to which the heat generated from the entrance end 31 is transmitted. Considering the heat transmission efficiency from the entrance end 31 to the first heat transmission member 170, the first heat transmission member 170 is preferably arranged between the light-collecting optical system 107 and the light source-side connection port 101 in the optical axis direction. As illustrated in FIG. 5, the first heat transmission member 170 may extend further inward than the entrance end 31 in the insertion direction of the endoscope-side connector 27.

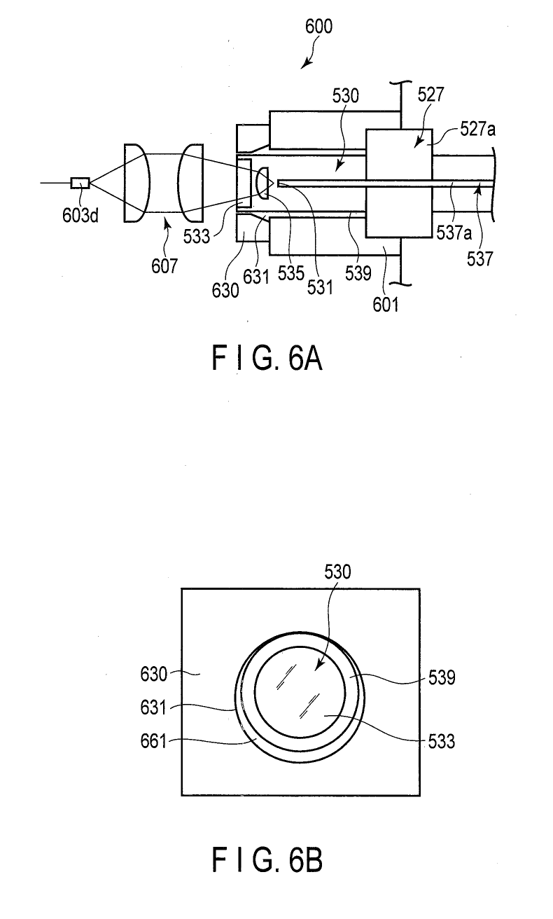

FIG. 6A and FIG. 6B illustrate a general endoscope-side connector 527, an optical connection portion 530, a light source device 600, a light source-side connection port 601, and a positioning member 630.

The general light source device 600 includes the positioning member 630 that positions the optical connection portion 530 with respect to an exit end 603d when the endoscope-side connector 527 is connected to the light source-side connection port 601. The positioning member 630 includes an insertion hole 631 into which the optical connection portion 530 is inserted. By inserting the optical connection portion 530 into the insertion hole 631, the optical connection portion 530 is positioned with respect to the exit end 603d. The inner diameter of the insertion hole 631 is larger than the outer diameter of the housing 539 provided in the optical connection portion 530. Therefore, a fourth space 661 is formed between the optical connection portion 530 and the insertion hole 631. The resistance is reduced between the optical connection portion 530 and the insertion hole 631 by the fourth space 661, but the optical connection portion 530 rattles against the insertion hole 631. As a result, the optical connection portion 530 is displaced with respect to the exit end 603d.

When an object is observed, the primary light, which is laser light, is radiated from the exit end 603d, and enters the entrance end 531 of the light guide 537 through the light-collecting optical system 607, the cover glass 533, and the lens 535. However, in general, the core diameter of the optical fiber 537a, which is an example of the light guide 537, is as small as 50 μm to 300 μm. Therefore, even if the optical connection portion 530 is positioned with respect to the exit end 603d, part of the primary light does not enter the core (entrance end 531) of the optical fiber 537a. As described above, if the optical connection portion 530 is displaced with respect to the exit end 603d, the primary light that does not enter the core of the optical fiber 537a increases.

Moreover, part of the primary light is reflected by a cladding of the optical fiber 537a or a cover glass 533 arranged at the periphery of the optical fiber 537a, and it is absorbed by a peripheral member arranged at the periphery of the core, and converted into heat. Other parts of the primary light are scattered by the light-collecting optical system 607, the cover glass 533, the lens 535, or the like, and it is absorbed by the peripheral member, and converted into heat. In particular, since the light intensity is high at the collected point of the primary light as the laser light, unless the primary light enters the core, heat is locally generated at the peripheral member.

As a result, the temperature at the periphery of the entrance end 531 rises. If the temperature rises significantly, the internal parts of the optical connection portion 530 are damaged by heat. Examples of the internal parts include a light guide 537 and a fixing member (not shown) such as an adhesive that fixes the light guide 537 to the housing 539. The heat is transmitted to the endoscope-side connector 527 from the optical connection portion 530. Therefore, if the user touches the endoscope-side connector 527 to remove it from the light source device 600 before the endoscope-side connector 527 cools down sufficiently, the user might get burned.

The heat paths that transmits heat includes a first heat path from the optical connection portion 530 to the endoscope-side connector 527, a second heat path from the optical connection portion 530 to the positioning member 630, and a third heat path from the optical connection portion 530 to the peripheral space of the optical connection portion 530. In the first heat path, the distance from the entrance end 531 to the endoscope-side connector 527 is long, and an endoscope-side housing 527a of the endoscope-side connector 527 has a resin. For this reason, the heat resistance is increased, the heat transmission efficiency from the entrance end 531 to the endoscope-side connector 527 is low, and the heat release efficiency from the endoscope-side connector 527 to the peripheral space of the endoscope-side connector 527 is low. In other words, the heat is accumulated, and the user might get burned as described above. In the second heat path, the rattling optical connection portion 530 is brought into point contact with the positioning member 630. For this reason, the heat resistance is increased, the heat transmission efficiency from the optical connection portion 530 to the positioning member 630 is low, and as a result, the heat release efficiency from the positioning member 630 to the peripheral space of the positioning member 630 is low. In the third heat path, the heat release efficiency from the optical connection portion 530 to the peripheral space of the optical connection portion 530 depends on the surface area of the optical connection portion 530, and the surface area of the optical connection portion 530 is affected by the size of the optical connection portion 530. The size of the optical connection portion 530 is limited in consideration of the arrangement of the optical connection portion 530 with respect to the endoscope. Thus, it is difficult to greatly improve the heat release efficiency.

In the present embodiment, the optical connection portion 30 is positioned by the positioning member 130, is pressed on the positioning member 130 by the pressing member 140, and is pinched between the pressing member 140 and the positioning member 130. Therefore, the optical connection portion 30 does not rattle with respect to the exit end 103d, and is prevented from being displaced with respect to the exit end 103d. In other words, the optical connecting efficiency of the optical connection portion 30 with respect to the exit end 103d improves. Therefore, it is possible to cause the primary light to enter the light guide 37 from the entrance end 31, to suppress generation of heat at the periphery of the entrance end 31, to reduce the temperature rise of the endoscope-side connector 27 including the optical connection portion 30, and to reduce the waste of the primary light.

Even if part of the primary light does not enter the light guide 37 from the entrance end 31 and heat is generated at the periphery of the entrance end 31, the heat is transmitted to the heat transmission main body 171 from the optical connection portion 30 through the contact portion 173, and the heat is released from the heat transmission main body 171 to the peripheral space of the heat transmission main body 171. The heat is transmitted from the heat transmission main body 171 to the first heat release portion 175, and is released from the first heat release portion 175 to the peripheral space of the first heat release portion 175. Since the first cooler 180 blows wind to the first heat release portion 175, for example, the heat transmission efficiency from the first heat release portion 175 to the peripheral space of the first heat release portion 175 further improves. In this manner, the heat is transmitted from the optical connection portion 30 to the peripheral space of the optical connection portion 30 by the first heat transmission member 170. Therefore, it is possible to prevent the heat from being accumulated in the optical connection portion 30, to reduce the temperature rise at the periphery of the entrance end 31, and to prevent the internal members of the optical connection portion 30 from being damaged by heat. Examples of the internal members include a light guide 37 and a fixing member (not shown) such as an adhesive that fixes the light guide 37 to the housing 39. It is also possible to prevent the heat from being transmitted to the endoscope-side connector 27 from the optical connection portion 30. Thus, the temperature of the endoscope-side connector 27 does not rise to the temperature at which the user gets burned. In other words, if the user touches the endoscope-side connector 27 to remove it from the light source device 100 before the endoscope-side connector 27 cools down sufficiently, the user can be prevented from getting burned. In this manner, it is possible to reduce the temperature rise of the endoscope-side connector 27 including the optical connection portion 30, and to prevent the temperature of the endoscope-side connector 27 including the optical connection portion 30 from reaching the intended temperature or higher. In other words, the temperature of the endoscope-side connector 27 including the optical connection portion 30 can be maintained below an intended temperature. In addition, it is possible to prevent problems in removal of the endoscope-side connector 27 caused by the temperature rise (for example, burning the user).

It is assumed that part of the primary light does not enter the entrance end 31 due to the positional shift of the optical connection portion 30 with respect to the exit end 103d. Furthermore, it is assumed that part of the primary light is scattered or reflected by the cover glass 533 or the like. In this case, since the light intensity is high at the collected point of the primary light as the laser light, high heat locally occurs. In the present embodiment, the pressing member 140 and the positioning member 130 can prevent the positional deviation of the optical connection portion 30 with respect to the exit end 103d, thereby improving the optical coupling efficiency of the optical connection portion 30 with respect to the exit end 103d. In addition, it is possible to suppress the primary light that does not enter the entrance end 31, to suppress generation of heat, and to eliminate the waste of the primary light.

The first heat transmission member 170 transmits heat from the optical connection portion 30 to the peripheral space of the optical connection portion 30 by the heat transmission main body 171, the contact portion 173, and the first heat release portion 175. Therefore, heat can be efficiently released from the optical connection portion 30, and the temperature rise of the optical connection portion 30 can be reduced.

The first heat path that transmits heat in this embodiment may be a path from the optical connection portion 30 to the endoscope-side connector 27. In this case, the distance from the entrance end 31, which is the portion where heat is generated most in the optical connection portion 30, to the endoscope-side connector 27 is long, and an endoscope-side housing 27a of the endoscope-side connector 27 has a resin. For this reason, the heat resistance is increased, the heat transmission efficiency from the entrance end 31 to the endoscope-side connector 27 is low, and the heat release efficiency from the endoscope-side connector 27 to the peripheral space of the endoscope-side connector 27 is low. In this case, the heat tends to be accumulated in the optical connection portion 30. However, according to the present embodiment, the heat is transmitted from the optical connection portion 30 to the peripheral space of the optical connection portion 30 by the first heat transmission member 170. For this reason, it is possible to prevent the heat from being accumulated in the optical connection portion 30.

When the pressing member 140 presses the optical connection portion 30, the contact portion 173 is deformed in conformity with the shape of the outer peripheral surface of the optical connection portion 30, and brought into close contact with the outer peripheral surface of the optical connection portion 30. The contact portion 173 is brought into surface contact. Thus, the contact portion 173 can prevent the optical connection portion 30 from being damaged by pressing, and can prevent the optical connection portion 30 from rattling. Moreover, it is possible to reliably prevent the positional deviation of the optical connection portion 30 with respect to the exit end 103d, thereby reliably improving the optical coupling efficiency of the optical connection portion 30 with respect to the exit end 103d. The contact portion 173 is thermally and directly connected to the optical connection portion 30. For this reason, the heat generated from the optical connection portion 30 can be efficiently transmitted to the first heat transmission member 170 through the contact portion 173. As a result, in the second heat path of the present embodiment from the optical connection portion 30 to the first heat transmission member 170, it is possible to increase the heat release efficiency from the first heat transmission member 170 to the peripheral space of the first heat transmission member 170. Furthermore, in the first example of the first heat transmission member 170, for example, it is possible to increase the heat release efficiency from the positioning member 130, which may have a member with high heat conductivity, to the peripheral space of the positioning member 130.

By the first heat release portion 175, it is possible to prevent the heat generated from the optical connection portion 30 from being accumulated in the first heat transmission member 170, and to efficiently release the heat to the peripheral space of the first heat release portion 175.

Since the light intensity is high at the collected point of the primary light as the laser light, high heat locally occurs. Therefore, heat is generated around the entrance end 31. In the present embodiment, the first heat transmission member 170 is arranged at least at the periphery of the entrance end 31 including the entrance end 31, which is a portion where heat is most generated in the optical connection portion 30. Therefore, high heat locally generated from the periphery of the entrance end 31 including the entrance end 31 can be efficiently transmitted to the first heat transmission member 170, and the temperature rise of the optical connection portion 30 can be reduced. Furthermore, even if the optical function of the endoscope 20 is different from the optical function of other endoscope, the temperature rise of the optical connection portion 30 can be reduced with respect to the optical connection portion 30 of any type of the endoscope 20. Moreover, it is possible to reduce the temperature rise in the optical connection portion 30 without being affected by the length of the optical connection portion 30.

Since the positioning member 130 is brought into surface contact with the optical connection portion 30, it can reliably position the optical connection portion 30. Since the positioning member 130 is brought into point contact with the optical connection portion 30, it can reliably position the optical connection portion 30.

The pressing member 140 faces the positioning member 130 in the first orthogonal direction. Thus, the optical connection portion 30 can be pinched between the pressing member 140 and the positioning member 130. Therefore, it is possible to prevent rattling of the optical connection portion 30 with respect to the exit end 103d, and to prevent the positional deviation of the optical connection portion 30 with respect to the exit end 103d.

The switching mechanism 150 switches between the pressed state and the released state. Therefore, it is possible to easily perform pressing in conjunction with the attachment of the optical connection portion 30 to the light source device 100, and to easily release the pressing in conjunction with the removal of the optical connection portion 30 from the light source device 100. Furthermore, it is possible to reduce the effort and stress on the user in performing pressing or releasing the pressing.

Since the first heat transmission member 170 functions as at least one of the positioning member 130 and the pressing member 140, it is unnecessary to independently arrange the first heat transmission member 170 itself. Therefore, the number of components can be reduced, the internal space of the light source device 100 can be suppressed, and the cost of the light source device 100 can be reduced.

The third heat path that transmits heat in the present embodiment may be a path from the optical connection portion 30 to the peripheral space of the optical connection portion 30. The heat release efficiency from the optical connection portion 30 to the peripheral space of the optical connection portion 30 depends on the surface area of the optical connection portion 30, and the surface area of the optical connection portion 30 is affected by the size of the optical connection portion 30. The size of the optical connection portion 30 is limited in consideration of the arrangement of the optical connection portion 30 with respect to the endoscope 20. Thus, it is difficult to greatly improve the heat release efficiency in only the optical connection portion 30. However, in the present embodiment, with the first heat transmission member 170, it is unnecessary to consider the surface area of the optical connection portion 30 with respect to the heat release efficiency, and it is possible to easily improve the heat release efficiency without influence of the size of the optical connection portion 30.

It is expected that the light quantity of the primary light radiated from the exit end 103d increases due to the high frame rate of imaging by the endoscope 20. In this case, when the optical connection portion 30 is displaced with respect to the exit end 103d, for example, the temperature of the heat locally generated at the periphery of the entrance end 31 becomes high. However, in the present embodiment, it is possible to suppress the generation of local heat by the positioning member 130, the pressing member 140, and the first heat transmission member 170, and to prevent the internal member of the optical connection portion 30 from being damaged by heat. In addition, since the temperature of the endoscope-side connector 27 does not become high, even if the user touches the endoscope-side connector 27 to remove it from the light source device 100, the user can be prevented from being burned.

### Modification 1 of First Embodiment

In the following, mainly the configurations different from those of the first embodiment will be described.

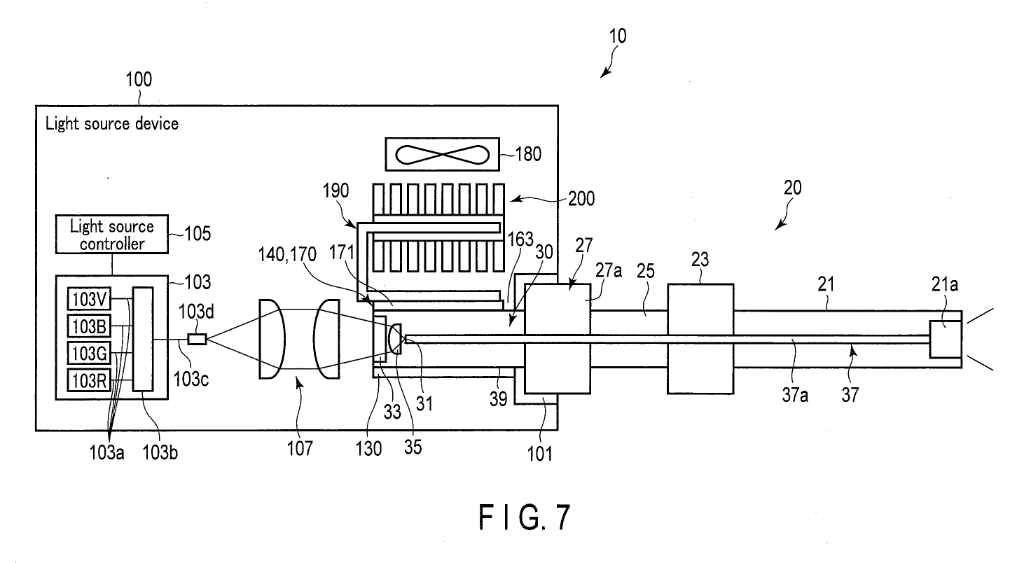

As illustrated in FIG. 7, the first heat release portion 175 is omitted.

As illustrated in FIG. 7, the light source device 100 includes a heat transport mechanism 190 that is connected to the first heat transmission member 170 and transports the heat transmitted from the first heat transmission member 170, and a second heat release portion 200 that is connected to the heat transport mechanism 190.

The heat transport mechanism 190 includes one end portion connected to an upper surface of the heat transmission main body 171, and the other end portion connected to the second heat release portion 200. It is preferable that one end portion is connected to at least the periphery of the entrance end 31 including the entrance end 31, which is the portion where heat is generated most. Thus, the heat transport mechanism 190 is thermally and directly connected to the first heat transmission member 170 at one end portion, and is thermally and directly connected to the second heat release portion 200 at the other end portion. The heat transport mechanism 190 can transport the heat transmitted from the heat transmission main body 171 to a position away from the first heat transmission member 170. The arrangement position of the heat transport mechanism 190 is not particularly limited. The heat transport mechanism 190 transports the heat transmitted from the heat transmission main body 171 to the second heat release portion 200 from the heat transport mechanism 190. The heat transport mechanism 190 includes, for example, a heat pipe.

The second heat release portion 200 releases the heat transported from the heat transport mechanism 190 to the second heat release portion 200 to a peripheral space of the second heat release portion 200 from the second heat release portion 200. The second heat release portion 200 may function as, for example, a heat sink. The second heat release portion 200 includes, for example, pin-shaped metal members. The metal members are arranged, for example, along the first orthogonal direction. The metal members are, for example, arranged at equal intervals with respect to each other in the insertion/removal direction, and the second orthogonal direction. By the metal members, the surface area of the second heat release portion increases. Therefore, the heat release ability of the second heat release portion 200 improves, and the heat transmission efficiency from the second heat release portion 200 to the peripheral space of the second heat release portion 200 improves.

A first cooler 180 of the present modification includes an air cooler such as a fan that provides wind toward the second heat release portion 200. The first cooler 180 further improves the heat transmission efficiency of the heat from the second heat release portion 200 to the peripheral space of the second heat release portion 200. In FIG. 7, the first cooler 180 is arranged above the second heat release portion 200 in the orthogonal direction. However, the arrangement position of the first cooler 180 can be appropriately changed as desired, for example, in accordance with the heat release ability of the second heat release portion 200, the arrangement state of the components inside the light source device 100, and the like. The first cooler 180 may be, for example, arranged on the side of the second heat release portion 200.

In the configuration illustrated in FIG. 7, the first heat release portion 175 may be provided. In this case, the heat transport mechanism 190 is connected to the heat transmission main body 171 while avoiding the first heat release portion 175. For example, the heat transport mechanism 190 is connected to the side surface of the heat transmission main body 171.

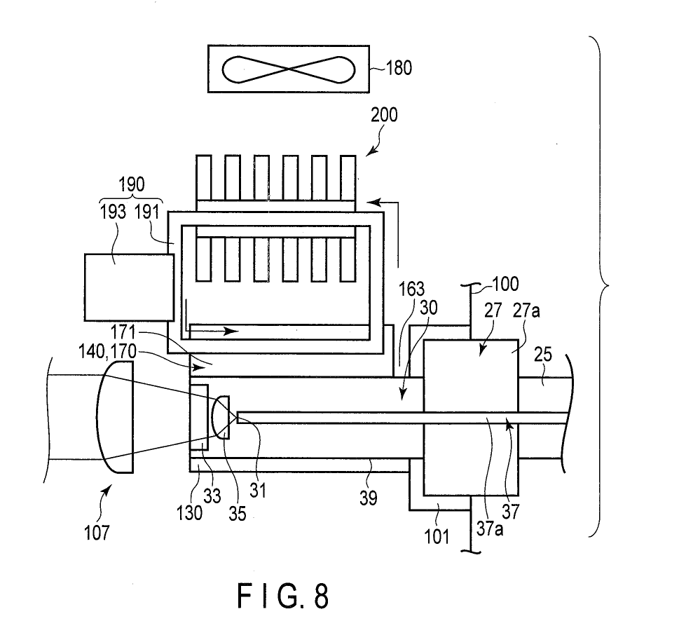

As illustrated in FIG. 8, the heat transport mechanism 190 may be arranged in a circulating manner and may have a flow path 191 through which a cooled fluid flows, and a circulator 193 to circulate the cooled fluid in the flow path 191. The cooled fluid is a cooled liquid, or a cooled gas. The cooled fluid is filled in the flow path 191. The flow path 191 is, for example, cylindrical. The circulator 193 is attached to the flow path 191, and the flow path 191 is connected to the side surface of the heat transmission main body 171 and the second heat release portion 200. The circulator 193 circulates the cooled fluid so that the cooled fluid is circulated through the flow path 191 in the order of the circulator 193, the first heat transmission member 170, the second heat release portion 200, and the circulator 193. The circulator 193 includes, for example, a pump and the like. The cooled fluid flowing through the flow path 191 absorbs heat generated from the first heat transmission member 170, and transmits the heat to the second heat release portion 200. The heat is released to the peripheral space from the flow path 191 when being transported by the cooled fluid flowing through the flow path 191. Furthermore, the heat is released from the second heat release portion 200 to the peripheral space of the second heat release portion 200.

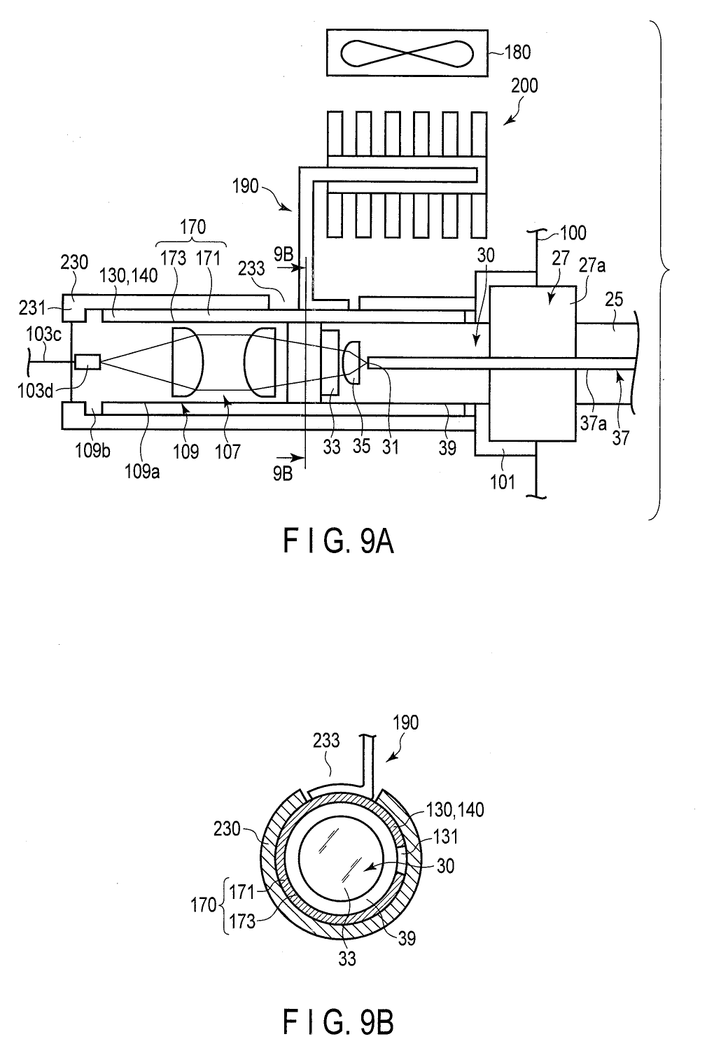

As illustrated in FIG. 9A, the light source device 100 may include a lens unit 109 having a lens housing 109a. The lens housing 109a holds the exit end 103d of the light guide 103c and the light-collecting optical system 107 in a state in which they are positioned with respect to each other. The lens housing 109a includes a convex portion 109b provided on the outer peripheral surface of the lens housing 109a. The convex portion 109b is provided at the end portion of the lens housing 109a arranged at an end portion side of the positioning member 130.

As illustrated in FIG. 9A and FIG. 9B, the positioning member 130 comprises a split sleeve that is elastically deformable so as to reduce its diameter in the radial direction of the positioning member 130. Such a positioning member 130 is, for example, a metal or a resin. The cross section of the positioning member 130 is arranged in a plane direction orthogonal to the longitudinal axis direction of the positioning member 130. The cross section has, for example, a C shape, and is continuous in the longitudinal axis direction of the positioning member 130. Such a positioning member 130 has a substantially cylindrical shape. The positioning member 130 is arranged along the longitudinal axis direction of the positioning member 130, and has a slit 131 that penetrates the positioning member 130 in the longitudinal axis direction. The slit 131 penetrates the positioning member 130 in the thickness direction of the positioning member 130.

The optical connection portion 30 and the lens unit 109 are press-fitted into the positioning member 130. The positioning member 130 presses the inner peripheral surface of the positioning member 130 on the outer peripheral surface of the housing 39 and the outer peripheral surface of the lens housing 109a, by the press fitting and the elastic deformation of the split sleeve. The inner peripheral surface of the positioning member 130 deforms in conformity with the shapes of the outer peripheral surface of the housing 39 and the outer peripheral surface of the lens housing 109a, and is in close contact with the outer peripheral surfaces. In this way, the positioning member 130 may function as the pressing member 140. In other words, the positioning member 130, the pressing member 140, and the first heat transmission member 170 are the same member. The inner peripheral surface of the positioning member 130 functions as the contact portion 173, and the positioning member 130 functions as the heat transmission main body 171. A sheet-like contact portion 173 as in the first embodiment may be provided on the inner peripheral surface of the positioning member 130.

The light source device 100 includes a fixing member 230 that fixes the positioning member 130 inside the light source device 100. The fixing member 230 is fixed to the inside of the light source device 100, and to the periphery of the light source-side connection port 101. The fixing member 230 is, for example, a sleeve guide. The fixing member 230 is, for example, a metal. The fixing member 230 has a substantially cylindrical shape. The inner shape of the fixing member 230 is substantially equal to the outer shape of the positioning member 130, and the inner diameter of the fixing member 230 is substantially equal to the outer diameter of the positioning member 130. When the positioning member 130 is inserted into the fixing member 230, the fixing member 230 is engaged with the positioning member 130, and the fixing member 230 positions and fixes the positioning member 130. The fixing member 230 has a bottom portion 231 provided at an end portion of the fixing member 230. The fixing member 230 has a cutout 233 formed on a part of the peripheral wall. The cutout 233 penetrates the peripheral wall in the thickness direction of the fixing member 230. The cutout 233 is arranged on the side of the optical connection portion 30 when the optical connection portion 30 is optically connected to the exit end 103d. The cutout 233 may be arranged on the side of the portion where heat is most generated in the optical connection portion 30, or on the side of the periphery of the above-described portion. The portion indicates, for example, the entrance end 31.

The inner shape of the positioning member 130 is substantially equal to the outer shape of the housing 39, and is substantially equal to the outer shape of the lens housing 109a. The inner diameter of the positioning member 130 is substantially equal to the outer diameter of the housing 39, and is substantially equal to the outer diameter of the lens housing 109a. The lens housing 109a is press-fitted into the positioning member 130 from one end portion of the positioning member 130 so that the convex portion 109b is pinched between the one end portion of the positioning member 130 and the bottom portion 231 of the fixing member 230. The positioning member 130 including the lens housing 109a is inserted into the fixing member 230 from the other end portion of the fixing member 230. As a result, the positioning member 130 including the lens unit 109 is engaged with the fixing member 230, and is positioned and fixed to the light source device 100. The housing 39 is press-fitted into the positioning member 130 from the other end portion of the positioning member 130, so that the entrance end 31 is optically connected to the exit end 103d of the light guide 103c. As a result, the optical connection portion 30 is engaged with the positioning member 130, is positioned and fixed to the light source device 100, and is optically connected to the exit end 103d of the light guide 103c.

The heat transport mechanism 190 includes a second heat transmission member that is a member having a high heat conductivity. The second heat transmission member is, for example, in the form of a sheet or belt. The second heat transmission member includes, for example, a graphite sheet. In the graphite sheet, the heat conductivity in the planar direction of the graphite sheet is higher than the heat conductivity in the thickness direction of the graphite sheet. The second heat transmission member has one end portion connected to the outer peripheral surface of the fixing member 230 through the cutout 233, and the other end portion connected to the second heat release portion 200. One end portion may be arranged on the side of the optical connection portion 30, for example, at the side of the portion where heat is most generated at the optical connection portion 30, or at the side of the periphery of the portion.

In this manner, the heat transport mechanism 190 may include at least one of the heat pipe, the flow path 191, and the second heat transmission member.

In some cases, an electrical connection portion (not shown) or the like is provided at the periphery of the light source-side connection port 101 inside the light source device 100, and the surface area of the first heat transmission member 170 may not be sufficiently secured. However, in the present modification, heat can be transported to a position distant from the first heat transmission member 170 by the heat transport mechanism 190. Therefore, in the present modification, the space for the first heat transmission member 170 can be minimized at the periphery of the light source-side connection port 101. Moreover, the present modification can improve a degree of freedom of design for heat release. Heat can be released by the heat transport mechanism 190 at a position distant from the optical connection portion 30. Therefore, the optical connection portion 30 can be prevented from being affected by the released heat.

The configuration illustrated in FIG. 9A can also release the heat in the lens housing 109a. In the configuration illustrated in FIG. 9A, the positioning member 130, the pressing member 140, and the first heat transmission member 170 are the same member. Therefore, the switching mechanism 150 can be made unnecessary, the number of components can be reduced, the internal space of the light source device 100 can be suppressed, the cost of the light source device 100 can be reduced, and the assembly can be simplified.

### Modification 2 of First Embodiment

In the following, mainly the configurations different from those of the first embodiment will be described.

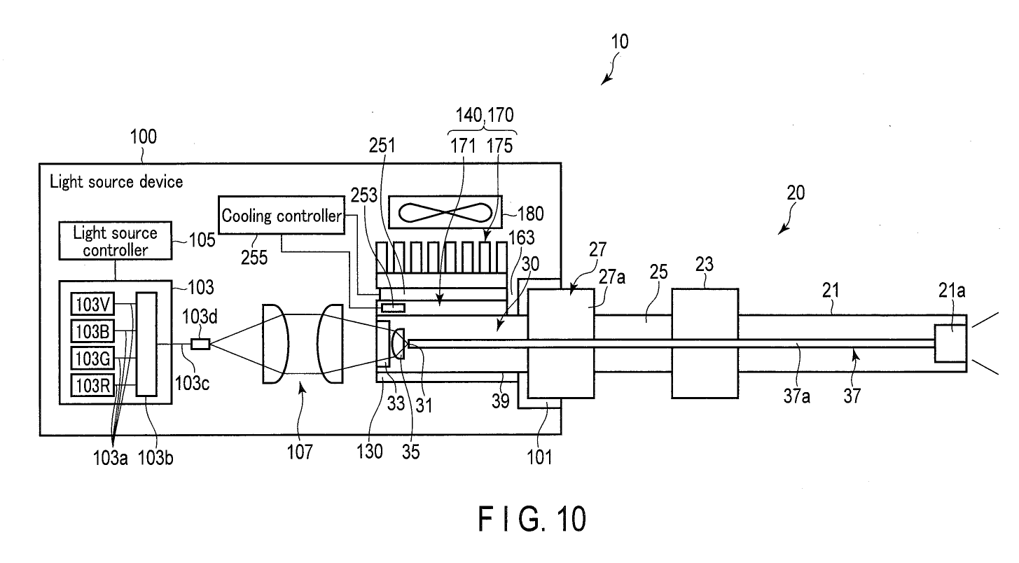

As illustrated in FIG. 10, the light source device 100 includes: a second cooler 251 arranged in the first heat transmission member 170 and configured to cool the first heat transmission member 170; a measurement device 253 measuring the temperature of, for example, the first heat transmission member 170, which is the measurement target portion; and a cooling controller 255 configured to control the driving of the second cooler 251 so that the temperature of the measurement target portion measured by the measurement device 253 becomes lower than an intended temperature. The first heat release portion 175 is arranged on the second cooler 251. The first heat release portion 175 is arranged on the side opposite to the heat transmission main body 171 with the second cooler 251 therebetween, in the first orthogonal direction.

The second cooler 251 is, for example, arranged in the heat transmission main body 171, and cools the optical connection portion 30 through the heat transmission main body 171. The second cooler 251 includes, for example, a Peltier element.

The measurement device 253 is arranged, for example, in the heat transmission main body 171. The measurement device 253 measures the temperature of the heat transmission main body 171, by regarding the temperature of the heat transmission main body 171 as the temperature of the optical connection portion 30. The measurement device 253, for example, measures the temperature of the heat transmission main body 171 when the light source section 103 is driven. The measurement target portion may be at least one of the optical connection portion 30 and the first heat transmission member 170. Thus, the measurement device 253 is thermally connected to the optical connection portion 30 when the optical connection portion 30 is attached to the light source device 100, and the measurement device 253 may measure the temperature of the optical connection portion 30. The measurement device 253 includes, for example, a temperature sensor.

The measurement device 253 may be arranged in the optical connection portion 30. The measurement device 253 may output the measurement result to the cooling controller 255 through an electrical connection unit (not shown) provided in the light source device 100, when the optical connection portion 30 is attached to the light source device 100. The measurement device 253 may always perform measurement, or may start measurement upon receipt of an instruction from the cooling controller 255 when the endoscope 20 is attached to the light source device 100.

The cooling controller 255 drives the second cooler 251 when the temperature of the measurement target portion reaches an intended temperature or higher. The intended temperature is, for example, a temperature undesirable to a user of the endoscope system 10 or to the endoscope-side connector 27 including the optical connection portion 30. The cooling controller 255 drives the second cooler 251 so that the temperature becomes lower than the intended temperature. If the temperature becomes lower than the intended temperature, the cooling controller 255 stops the driving of the second cooler 251. The cooling controller 255 is constituted by a hardware circuit including an ASIC or the like. The cooling controller 255 may be constituted by a processor. If the cooling controller 255 is constituted by a processor, a program code for causing the processor to function as the cooling controller 255 by execution of the processor, has been stored in an internal memory or an external memory (not shown) accessible by the processor.

In the present modification, the measurement device 253 measures the temperature of the measurement target portion, and the second cooler 251 cools the measurement target so that the temperature of the measurement target portion becomes lower than an intended temperature. Thus, it is possible to reliably suppress the internal member of the optical connection portion 30 from being damaged by heat, and to reliably suppress the heat from being transmitted from the optical connection portion 30 to the endoscope-side connector 27. In addition, even if the user touches the endoscope-side connector 27 to remove it from the light source device 100, the user can reliably be prevented from getting burned.

In the present modification, the cooling controller 255 may control the driving of the first cooler 180 based on the measurement result. For example, the cooling controller 255 drives the first cooler 180 when the temperature of the measurement target reaches the intended temperature or higher. The cooling controller 255 drives the first cooler 180 so that the temperature becomes lower than the intended temperature. If the temperature becomes lower than the intended temperature, the cooling controller 255 stops the driving of the first cooler 180.

The measurement device 253 and the cooling controller 255 of the present modification may be incorporated into the configuration of the first embodiment. In this case, the cooling controller 255 drives the first cooler 180 as described above.

Moreover, the cooling controller 255 may control the first cooler 180 without influence of the measurement result so that the first cooler 180 is driven when the endoscope-side connector 27 is inserted into the light source-side connection port 101, and that the first cooler 180 stops when the endoscope-side connector 27 is removed from the light source-side connection port 101.

### Second Embodiment

In the present embodiment, only the configurations different from those of the first embodiment and its modifications will be described.

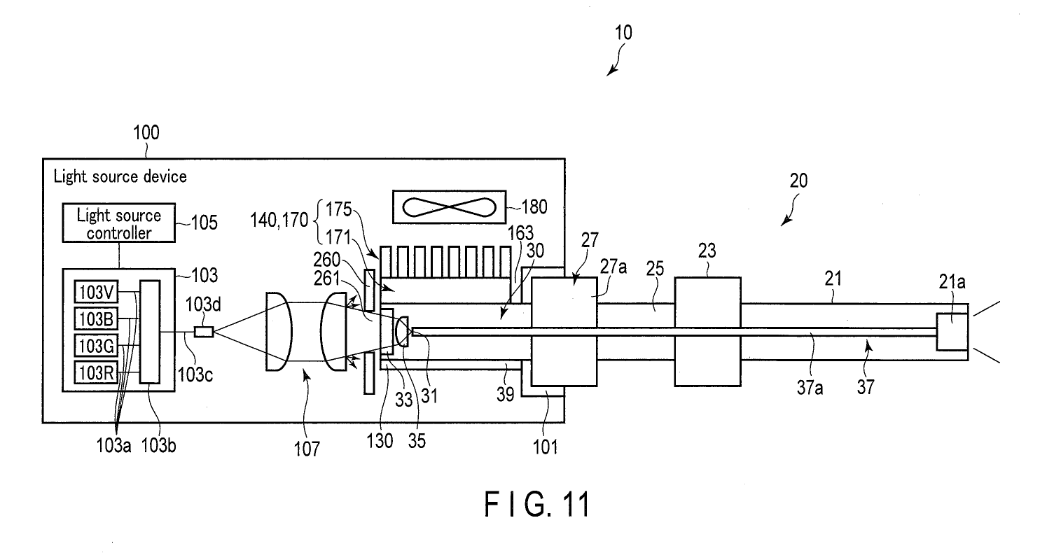

As illustrated in FIG. 11, the light source device 100 includes a light shield 260 that shields the primary light traveling to portions other than the entrance end 31, so as to release the heat generated from the shield primary light to portions other than the optical connection portion 30. The light shield 260 may release the heat to portions other than the optical connection portion 30 and the first heat transmission member 170. The light shield 260 has an opening 261 through which the primary light traveling from the light-collecting optical system 107 to the entrance end 31 can pass. The opening 261 has a diameter substantially equal to the numerical aperture of the optical fiber 37. The light shield 260 shields the primary light traveling from the light-collecting optical system 107 to portions other than the entrance end 31. The arrangement positions of the opening 261 and the light shield 260 are not particularly limited, as long as the primary light traveling from the light-collecting optical system 107 to the entrance end 31 passes through the opening 261, and the light shield 260 shields the primary light traveling from the light-collecting optical system 107 to portions other than the entrance end 31. For example, the light shield 260 including the opening 261 is arranged between the light-collecting optical system 107 and the cover glass 33 in the longitudinal axis direction of the optical connection portion 30. The light shield 260 is thermally separated from the optical connection portion 30 and the first heat transmission member 170. Therefore, the light shield 260 may be arranged separately from the optical connection portion 30 and the first heat transmission member 170. Alternatively, a member having low heat conductivity may be arranged between the light shield 260 and the optical connection portion 30, and between the light shield 260 and the first heat transmission member 170. The light shield 260 may be thermally connected to the first heat release portion 175.

The light shield 260 includes a member having high heat conductivity. The member is, for example, a metallic member, such as copper, aluminum, SUS, stainless steel, and aluminum nitride. The light shield 260 may include an uneven portion (not shown). The uneven portion is arranged on the surface on which the primary light is irradiated and that is the light-shielding region of the light shield 260. The uneven portion increases the surface area of the light-shielding region. Therefore, the heat release ability of the light shield 260 improves, and the heat transmission efficiency from the light shield 260 to the peripheral space of the light shield 260 improves. The light shield 260 may absorb the primary light.

Most of the primary light enters the entrance end 31, but a part of the primary light is scattered by the surface of the lens 35 of the light-collecting optical system 107. The scattered primary light tries to travel to the first heat transmission member 170 and the like. However, in the present modification, the light shield 260 shields the scattered primary light, absorbs the scattered primary light, and releases heat. The light shield 260 is thermally separated from the optical connection portion 30 and the first heat transmission member 170. Thus, the heat is not transmitted to the optical connection portion 30 and the first heat transmission member 170 from the light shield 260, and is released from the light shield 260 to the peripheral space of the light shield 260.

The present modification can reduce the temperature rise of the optical connection portion 30 and the first heat transmission member 170 by the scattered primary light.

### Modification 1 of Second Embodiment

In the following, mainly the configurations different from those of the second embodiment will be described.

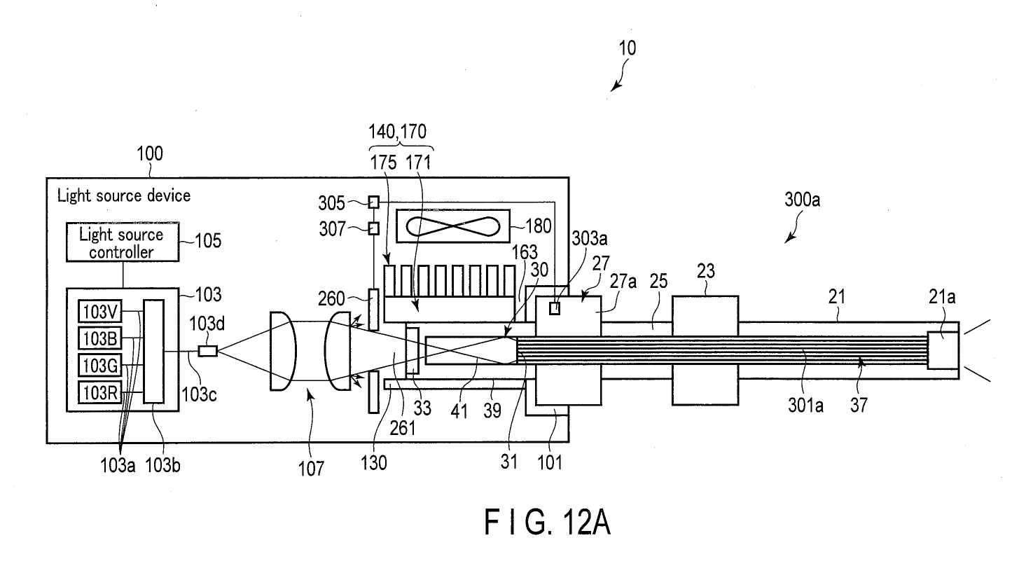

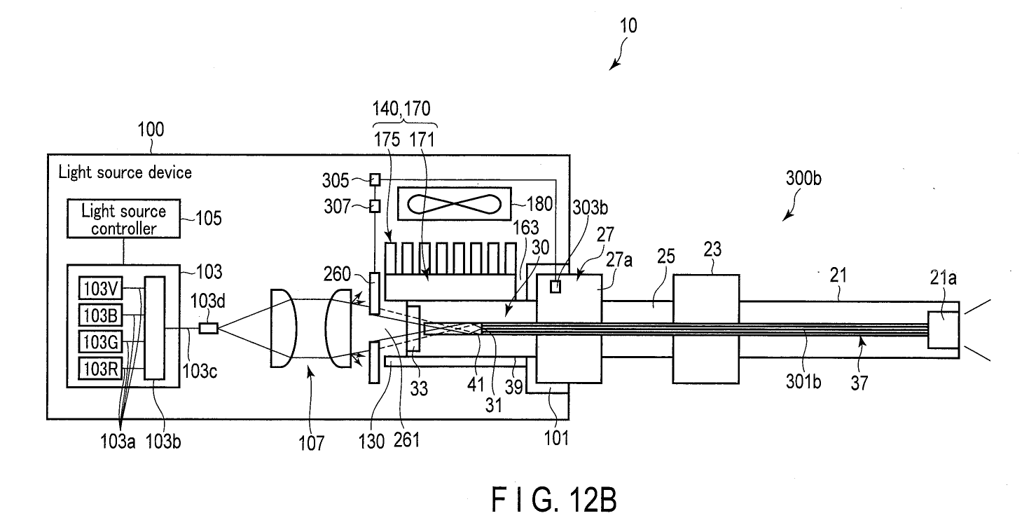

The light source device 100 needs to be shared and standardized for various types of endoscopes having different optical functions, for example, and it is necessary to be a general member. The optical function refers to, for example, characteristics of the light guide 37. Here, as an example of the optical function, an explanation will be given using first and second bundle fibers 301a and 301b, which are each an example of the light guide 37 as shown in FIG. 12A and FIG. 12B. In the following, an endoscope including the first bundle fiber 301a and an endoscope including the second bundle fiber 301b will be referred to as a first endoscope 300a and a second endoscope 300b, respectively.

As illustrated in FIG. 12A, for example, the first endoscope 300a includes the first bundle fiber 301a having a first diameter. The first endoscope 300a includes a first storage 303a that stores first information indicating that the endoscope is the first endoscope 300a. When the endoscope-side connector 27 is connected to the light source-side connection port 101, the first storage 303a transmits the first information to a determination unit 305 arranged in the light source device 100.

As illustrated in FIG. 12B, for example, the second endoscope 300b includes the second bundle fiber 301b having a second diameter smaller than the first diameter. The second endoscope 300b includes a second storage 303b that stores second information indicating that the endoscope is the second endoscope 300b. When the endoscope-side connector 27 is connected to the light source-side connection port 101, the second storage 303b transmits the second information to the determination unit 305.

The light source device 100 further includes the determination unit 305 that determines, based on the first information or the second information, whether the endoscope connected to the light source device 100 is the first endoscope 300a or the second endoscope 300b.

The light source device 100 further includes a light shielding controller 307 that controls the light-shielding area of the light shield 260 based on the determination result of the determination unit 305. For example, the light shielding controller 307 controls the light-shielding region of the light shield 260 so that the opening 261 expands or contracts in accordance with the optical function of the endoscopes 300a and 300b. The light-shielding region is controlled in accordance with the size of the opening. In this case, the light shield 260 includes a stop blade in which enlargement or reduction of the opening 261 is controlled by the light shielding controller 307. Alternatively, for example, the light shielding controller 307 may move the light shield 260 along the optical axis direction, in a state in which the size of the opening 261 is constant without enlarging or reducing the opening 261. Therefore, the light-shielding region is controlled in accordance with the position of the light shield 260. In this manner, the light shield 260 changes the light-shielding region in accordance with the optical function of the endoscope 20 connected to the light source device 100.

For example, when the first endoscope 300a is connected to the light source device 100, the light shielding controller 307 controls the light-shielding region of the light shield 260 so that the opening 261 expands. For example, when the second endoscope 300b is connected to the light source device 100, the light shielding controller 307 controls the light-shielding region of the light shield 260 so that the opening 261 contracts. The light shielding controller 307 is constituted by a hardware circuit including an ASIC or the like. The light shielding controller 307 may be constituted by a processor. If the light shielding controller 307 is constituted by a processor, a program code for causing the processor to function as the light shielding controller 307 by execution of the processor, has been stored in an internal memory or an external memory (not shown) accessible by the processor.

Depending on the optical function, the position of the entrance end 31 with respect to the exit end 103d differs. However, depending on the design conditions such as the internal space of the light source device 100, the light source device 100 may have to perform average performance on various types of endoscopes 20 in some cases. For example, when the second endoscope 300b is connected to the light source device 100, a part of the primary light indicated by a dotted line in FIG. 12B tends to be shaded by a cover glass 33 or the like, which is a peripheral member of the entrance end 31. The shaded primary light is absorbed by the optical connection portion 30 and converted into heat, and the optical connection portion 30 generates heat.

In the present modification, the light-shielding region of the light shield 260 is changed in accordance with the optical function, and the light shield 260 shields primary light that will be shaded in advance. Accordingly, it is possible to prevent heat generation by the optical connection portion 30.

The present invention is not limited to the above embodiments as they are, and can be embodied by modifying structural elements in the implementation stage without departing from the gist thereof. Further, various inventions can be formed by appropriately combining a plurality of structural elements disclosed in the above embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope light source device to which an optical connection portion of an endoscope is detachably attached, the optical connection portion including an entrance end, the endoscope light source device comprising:
a light source section that radiates primary light that enters the entrance end;
a positioning member that is fixed to the endoscope light source device, and positions the entrance end on an optical axis that is a center axis of the primary light radiated from the light source section when the optical connection portion is arranged in the endoscope light source device;
a pressing member that presses the optical connection portion toward the positioning member after the optical connection portion is arranged in the positioning member; and
a first heat transmission member that is capable of functioning as at least one of the positioning member and the pressing member and transmits heat generated from the optical connection portion when the optical connection portion is arranged in the endoscope light source device.

2. The endoscope light source device according to claim 1, wherein the first heat transmission member includes a contact portion that is arranged on a contact part with respect to the optical connection portion, and is deformed and brought into close contact with the optical connection portion when the pressing member presses the optical connection portion.

3. The endoscope light source device according to claim 2, wherein the contact portion is brought into surface contact.

4. The endoscope light source device according to claim 2, wherein the contact portion is thermally and directly connected to the optical connection portion.

5. The endoscope light source device according to claim 2, wherein the first heat transmission member includes a heat release portion that releases the heat transmitted from the contact portion to a peripheral space.

6. The endoscope light source device according to claim 1, wherein the first heat transmission member is arranged at least at a periphery of a portion including a part where the heat is most generated in the optical connection portion.

7. The endoscope light source device according to claim 1, wherein the positioning member is brought into surface contact or point contact with the optical connection portion.

8. The endoscope light source device according to claim 1, wherein the pressing member faces the positioning member in a first orthogonal direction orthogonal to an insertion/removal direction of the optical connection portion into/from the endoscope light source device.

9. The endoscope light source device according to claim 1, comprising a switching mechanism that switches between a pressed state in which the pressing member presses the optical connection portion toward the positioning member when the optical connection portion is attached to the endoscope light source device, and a released state in which the pressing member releases pressing against the optical connection portion when the optical connection portion is removed from the light source device.

10. The endoscope light source device according to claim 1, wherein the positioning member comprises a split sleeve into which the optical connection portion is press-fitted, and functions as the pressing member.

11. The endoscope light source device according to claim 1, comprising:
a heat transport mechanism that is connected to the first heat transmission member and transports the heat transmitted from the first heat transmission member; and
a heat release portion that is connected to the heat transport mechanism and releases the heat transported from the heat transport mechanism to a peripheral space.

12. The endoscope light source device according to claim 11, wherein the heat transport mechanism is thermally and directly connected to the first heat transmission member.

13. The endoscope light source device according to claim 12, wherein the heat transport mechanism comprises at least one of a heat pipe, a flow path through which a cooled fluid flows, and a second heat transmission member.

14. The endoscope light source device according to claim 1, comprising:
a cooler that cools the first heat transmission member;
a measurement device that measures a temperature of a measurement target portion that is at least one of the optical connection portion and the first heat transmission member; and
a cooling controller that controls driving of the cooler so that the temperature of the measurement target portion measured by the measurement device becomes lower than an intended temperature.

15. The endoscope light source device according to claim 1, comprising a light shield that shields the primary light traveling to portions other than the entrance end, so as to release the heat generated from the light-shielded primary light to portions other than the optical connection portion.

16. The endoscope light source device according to claim 15, wherein the light shield changes a light-shielding region in accordance with an optical function of the endoscope connected to the endoscope light source device.

17. An endoscope detachably attached to the endoscope light source device according to claim 1.

18. An endoscope system, comprising:
the endoscope light source device according to claim 1; and
an endoscope detachably attached to the endoscope light source device.
